(19) United States
(12) Patent Application Publication
(10) Pub. No.: US 2010/0016657 A1
(43) Pub. Date: Jan. 21, 2010

(54) Method and Apparatus for Managing Erectile Dysfunction

(75) Inventors: Peter Robertson, New South Wales (AU); Anthony Clyde Neason Stephen, New South Wales (AU)

Correspondence Address: JONES DAY, 222 EAST 41ST ST, NEW YORK, NY, 10017, US

(73) Assignee: Continence Control Systems International Pty Ltd, Chatswood, New South Wales (AU)

(21) Appl. No.: 12/226,678

(22) PCT Filed: Apr. 24, 2007
(86) PCT No.: PCT/AU2007/000525

§ 371 Date: Jun. 18, 2009

(30) Foreign Application Priority Data
Apr. 24, 2006 (AU) 2006902107

(51) Int. Cl. A61F 5/00; A61N 1/00

(52) U.S. Cl. 600/41; 607/39

(57) ABSTRACT

The present invention relates to an apparatus and method for treating erectile dysfunction. One of the causes of erectile dysfunction is venous leakage, where one or more veins carrying the blood from the penis are unable to retain sufficient blood within the penis to maintain erection. Embodiments of the present invention propose implanting contractile tissue sphincters around the base of the penis and/or around one or more veins, and stimulating the contractile tissue to contract when an erection is required to be maintained. Stimulation is provided by an implantable stimulator.

U.S. Pat. No. 6,659,936 issued on 9 Dec. 2003, International Patent Application No. PCT/AU00/00925 filed on 4 Aug. 2000, Australian Provisional Application AU PQ2026 filed on 4 Aug. 1999, relate to the control of urinary incontinence.

International Patent Application No. PCT/AU2005/001698 filed on 8 Nov. 2005, Australian Provisional Application No. AU2004906393 filed on 8 Nov. 2004, relate to an implantable electrode arrangement.

International Patent Application No. PCT/AU2006/001301 filed on 4 Sep. 2006, Australian Provisional Application No. AU2005904830, filed on 2 Sep. 2005, relate to an implant for managing a medical condition.

Australian Provisional Application No. 2006902107 filed 24 Apr. 2006 relate to a method and apparatus for managing erectile dysfunction.

International Patent Application No. PCT/AU2006/000258 filed on 2 Mar. 2006, Australian Provisional Application No. 2005900957 filed on 2 Mar. 2005, relate to an improved method and device for managing urinary incontinence.

Each one of the above documents are incorporated herein by reference in their entirety.

### FIELD OF THE INVENTION

The present invention relates to a method and apparatus for facilitating erection and, more particularly, but not exclusively, to a method and apparatus for managing erectile dysfunction.

### BACKGROUND OF THE INVENTION

Erectile dysfunction (commonly known as “impotence”) is a common condition. There are number of different causes, some more treatable than others. It is known, for example, that one cause of erectile dysfunction is an inability for blood supply to the penis to initiate and/or maintain an erection. In some cases this may be treated with pharmaceuticals arranged to promote blood supply to the penis e.g. sildenafil.

While a number of conditions may be responsive to blood supply promoters, not all causes of erectile dysfunction will be. Where a major artery (e.g. the deep artery or dorsal artery or both) is occluded, for example, then blood supply promoters will not be effective or wholly effective. Further, where venous leakage is occurring, blood supply promoters may not be effective in maintaining an erection.

In such cases it may be possible to treat the condition by penile vascular surgery. The success rate for penile vascular surgery (particular long term) is low, however. The selection criteria for appropriate patients is also fairly severe, so that the number of patients who are determined to be suitable for surgery is also low. In the case of massive venous leakage (where many blood vessels are leaking blood from the penis) the surgery will generally be ineffective and patients with this condition are not selected for vascular surgery. In addition, as the surgery involves permanent or partial occlusion of blood vessels and or surrounding tissue to slow venous leakage, subsequent atrophy and tissue damage may occur, or the formation of collateral circulation, either of which may reduce the effectiveness of the penile vascular surgery.

In an earlier patent application, International Patent Application No. PCT/AU00/00925 (referred to above), a method and apparatus is proposed for treating urinary incontinence which includes the steps of forming a “neosphincter” from smooth muscle tissue taken from elsewhere in the patient's body, and wrapping the neosphincter around the urethra. An implantable stimulator provides an electrical signal to the neosphincter via an electrode or electrodes. The electrical signal stimulates the neosphincter to maintain tone about the urethra to reduce leaks from the bladder until the user wishes to urinate. A signal from a control device may cause the stimulator to stop providing the electrical signal to the neosphincter, to allow the neosphincter to relax and enable the individual to urinate. The stimulation may activate the muscle directly, or activate it through the excitation of nerve fibres that innervate the muscle.

### SUMMARY OF THE INVENTION

In accordance with a first aspect, the present invention provides an apparatus for facilitating an erection in a patient, the apparatus including a stimulator arranged to provide a signal for stimulation of contractile tissue, in order to facilitate an erection.

In an embodiment, the contractile tissue is positioned proximate the penis and on activation is arranged to constrict one or more blood vessels and/or the cavernosal bodies in order to facilitate retention of blood in the penis and the promotion/maintenance of an erection. The contractile tissue may be implanted about the penis (under the skin) around the cavernosal bodies so that on constriction it constricts the cavernosal bodies and blood vessel(s), such as the dorsal vein. The contractile tissue may be in the form of a ring about the penis. Alternatively or additionally, the contractile tissue may be placed about one or more blood vessels e.g. the dorsal vein and may be in the form of a small “wrap” about the one or more blood vessels. The contractile tissue may be placed at the root of penis or may be placed internally further back about the left and right crura. In an embodiment, the erection is facilitated by the contraction of the contractile tissue maintaining the retention of blood in the penis, so that once blood has flowed into the penis, it does not flow out at such a rate that the erection fails.

In an embodiment, the contractile tissue may have properties the same as or similar to smooth muscle tissue. In an embodiment, the contractile tissue is smooth muscle tissue. In an embodiment, the sphincter or wrap may be formed from the dartos muscle or from a conveniently located portion of vein from the patient. In an embodiment, the sphincter or wrap may be formed from muscle from the wall of the gastro-intestinal tract. In an embodiment, the smooth muscle tissue may be transplanted tissue taken from a donor, from elsewhere in the patient's body, or may have been grown externally.

In an embodiment, the signal provided by the stimulator is in the form of a pulse signal, arranged to maintain tone in the contractile tissue.

In an embodiment, the stimulator is arranged to provide a different stimulation signal or no stimulation signal, in order to allow the contractile tissue to relax. A controller, operable by the patient, may be provided to vary the stimulation signal.

In an embodiment, the signal is arranged to stimulate the contractile tissue through stimulation of nerve fibres innervating the contractile tissue by means of an implantable electrode.

An advantage of an embodiment of the invention is that it may be effective to facilitate erection in cases of venous leakage, and even massive venous leakage, which is presently practically untreatable. Further, there will generally be no requirement for complex vascular surgery (e.g. joining blood vessels to each other). Further, unlike vascular surgery, when the patient does not wish to have an erection, the stimulation may be reduced or turned off to the contractile tissue, causing it to relax so the venous blood outflow from the penis is unimpeded. Additionally, in an embodiment, the patient or supervising clinician can vary the stimulation to the contractile tissue and thus conveniently adjust the extent of constriction applied by the contractile tissue. This may be able to provide more effective therapy as the patient's needs change due to response to surgery, age or changes in pharmacological regime. Further, unlike penile implants which require mechanical transfer of fluid by using a small hand pump in the scrotum, one advantage of at least an embodiment of this invention is that there is no mechanical manipulation of the delicate skin of the scrotum, risking erosion and or infection. Further, rather than penile implants which add significant bulk to the penis even when flaccid, making them uncomfortable and awkward, in at least an embodiment of this invention it is intended that there be only little bulk implanted in the penis, being the stimulation electrode and the wrap of contractile tissue, so that the penis can be allowed to return to a fully relaxed state when contractile tissue is not activated.

As discussed above, there may be a number of other causes of erectile dysfunction than venous leakage. Arterial insufficiency, for example, may lead to an inability to provide sufficient blood flow into the penis to cause or maintain an erection. Often, a subject may display symptoms of venous leakage and arterial insufficiency. In an embodiment, the stimulator is also arranged to stimulate a nerve or nerves affecting arterial blood supply, advantageously stimulating arterial blood supply, or to provide electrical stimulation that results in vasodilation of the arteries that provide blood to the penis.

In International Patent Application No. PCT/AU2006/00258, referred to above, a method and apparatus are disclosed for neurostimulation of nerves in a patient's anatomy in order to treat conditions such as urinary incontinence and fecal incontinence.

In an embodiment of the present invention, the stimulator is arranged to provide a signal or signals to stimulate one or more nerves in a patient's anatomy to treat other conditions. In an embodiment, the stimulator is arranged for stimulation of nerves to address urinary incontinence, in an embodiment urge incontinence (in which there is an inappropriate urge to urinate). This condition is known to be treated by stimulation of the sensory nerves using a technique known as neuromodulation. In an embodiment of this invention, neuromodulation may be facilitated by stimulation of the Dorsal Penile Nerve (DPN), which is a division of the Pudendal Nerve.

In an embodiment, the stimulator is an implantable stimulator arranged to be implanted within a patient.

In an embodiment, the apparatus has the advantage that it may be used where other treatments have failed.

In accordance with a second aspect, the present invention provides a device for facilitating an erection, the device including implanted contractile tissue positioned proximate or within the penile anatomy and arranged to be stimulated to contract to facilitate an erection.

In an embodiment, the device is implanted in the form of ring about the penis, so that on constriction it constricts one or more veins and/or the cavernosal bodies.

In an embodiment the contractile tissue is implanted as a small wrap about one or more blood vessels e.g. the deep dorsal vein. Contraction of the tissue results in occlusion of the vein which facilitates erection.

In accordance with a third aspect, the present invention provides a controller for controlling an stimulator which is arranged to stimulate contractile tissue positioned proximate or within the penile anatomy in order to facilitate an erection, the controller including means for providing a signal to the stimulator to vary the stimulation provided by the stimulator.

The controller may be arranged to provide a signal which causes the implantable stimulator to vary the stimulation (or remove stimulation) to allow the contractile tissue to contract or relax.

In an embodiment, the controller is arranged to generate a wireless signal to be received by a receiver associated with the stimulator.

In an embodiment, the stimulator is an implantable stimulator, that is, it is implantable within the patient.

In accordance with a fourth aspect, the present invention provides a programmer unit arranged to adjust parameters of the apparatus of the first aspect of the present invention.

In accordance with a fifth aspect, the present invention provides a system for facilitating an erection, the system comprising an apparatus in accordance with the first aspect of the invention and a device in accordance with the second aspect.

In an embodiment, the system also includes a controller in accordance with the third aspect of the invention.

In an embodiment, the system also includes a programmer unit in accordance with the fourth aspect of the invention.

In accordance with a sixth aspect, the present invention includes a system for facilitating an erection, comprising an apparatus in accordance with the first aspect of the present invention and a controller in accordance with the third aspect of the present invention.

In accordance with a seventh aspect, the present invention provides a system for facilitating an erection, comprising a apparatus in accordance with a first aspect of the present invention and a programmer unit in accordance with the fourth aspect.

In accordance with an eighth aspect, the present invention provides a method of facilitating an erection, comprising the steps of stimulating contractile tissue positioned proximate or in the penile anatomy of a patient in order to cause the contractile tissue to contract, by way of providing a stimulation signal to an electrode to transmit the signal to the contractile tissue.

In an embodiment, the contractile tissue contracts and constricts in order to restrict venous blood flow and therefore maintain an erection.

In one embodiment, the method comprises the further step of providing a further signal, or absence of a signal, in order to enable the contractile tissue to relax.

In accordance with a ninth aspect, the present invention provides a method of facilitating an erection, comprising the steps of implanting into the patient a stimulator device arranged to provide stimulation signals to contractile tissue in order cause the tissue to contract to facilitate restriction of one or more blood vessels and/or the cavernosal bodies in order to facilitate maintaining blood within the penis.

In one embodiment, the method comprises the further step of implanting the contractile tissue.

In accordance with a tenth aspect, the present invention provides a method of facilitating an erection, comprising the steps of implanting contractile tissue in a position proximate to or within the penile anatomy, the contractile tissue being arranged to be stimulated to contract about one or more blood vessels and/or the cavernosal bodies in order to maintain blood within the penis in order to facilitate an erection.

In an embodiment, the erection is facilitated by being maintained because the blood is retained in the penis by the contracting action of the tissue. Blood flow away from the penis is therefore restricted.

In accordance with an eleventh aspect, the present invention provides an apparatus for facilitating an erection in a patient, the apparatus including a stimulator arranged to provide a signal for stimulating a nerve or nerves affecting the arterial blood supply to the penis.

In accordance with a twelfth aspect, the present invention provides a method of facilitating an erection in a patient, comprising the steps of stimulating a nerve or nerves to affect arterial blood supply to the penis.

In accordance with a thirteenth aspect, the present invention provides a method of treating a condition in a patient, comprising the steps of stimulating the Dorsal Penile Nerve in order to treat the condition.

In an embodiment, treatment is through neuromodulation, that is, using electrical stimulation in one neural pathway to modulate pre-existing activity in another.

In an embodiment, the condition is urinary incontinence, in an embodiment being urge incontinence.

In an embodiment, the condition is fecal incontinence.

### BRIEF DESCRIPTION OF THE DRAWINGS

Features and advantages of the present invention will become apparent from the following description of embodiments thereof, by way of example only, with reference to the accompanying drawings, in which:

FIG. 1 is a cross section through the male pelvic anatomy schematically showing positioning of an apparatus and device in accordance with one embodiment of the present invention;

FIG. 2 is a further section through the male pelvic anatomy showing positioning of an apparatus and device in accordance with a further embodiment of the present invention;

FIG. 3 is a further section through the male pelvic anatomy showing positioning of an apparatus and device in accordance with a further embodiment of the present invention;

FIG. 4 is a section through the male pelvic anatomy showing positioning of an apparatus and device in accordance with a further embodiment of the present invention;

FIG. 5 is a block diagram of components of an implantable stimulator in accordance with an embodiment of the present invention;

FIG. 6 is a block diagram showing components of a system in accordance with an embodiment of the present invention; and

FIG. 7 is a block diagram showing components of the system in accordance with an embodiment of the present invention;

FIGS. 8, 9 & 10 are exploded perspective, plan and side views, respectively, of an electrode arrangement for delivering stimulation signals in a system in accordance with an embodiment of the present invention;

FIGS. 11, 12, 13, 14 and 15 are perspective, plan, side section, detail views of a shroud component of the electrode arrangement of FIG. 7;

FIGS. 16, 17, 18, 19, 20, are perspective, rear, plan section, side section and plan views of a cover component of the electrode arrangement of FIG. 7

FIG. 21 is a further cross section through the male pelvic anatomy schematically showing positioning of an apparatus in accordance with a third embodiment of the present invention; and

FIG. 22 is a diagram illustrating interleaving signals of electrical stimulation in accordance with an embodiment of the present invention.

### DETAILED DESCRIPTION OF EMBODIMENTS

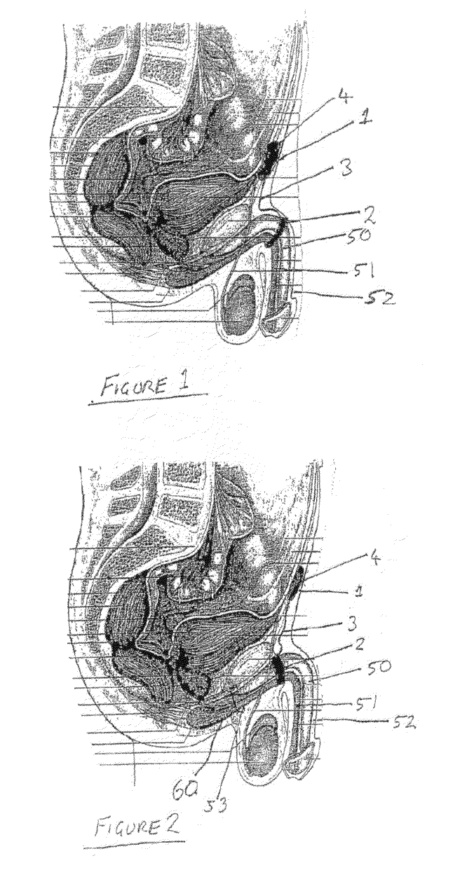

Referring to FIG. 1, a system in accordance with an embodiment of the present invention, for facilitating an erection, is shown. The system includes an apparatus comprising an implantable stimulator 1, and a device which comprises contractile tissue 2. The implantable stimulator 1, in this embodiment includes electronic circuitry which is arranged to provide an electrical signal for stimulating the contractile tissue 2. In this embodiment, an insulated conductor 3 conducts the signal or signals (one or more signals may be provided) to an electrode or electrodes implanted proximate or within the contractile tissue 2.

The contractile tissue in this embodiment is formed into a band which is placed at the root of the penis 52 about the cavernosal bodies 50 and corpus spongiosum 51. In operation, when the contractile tissue 2 is stimulated, it contracts about the cavernosal bodies 50 and corpus spongiosum 51. The cavernosal bodies carry important veins, in particular the dorsal vein of the penis 52. Contraction of the contractile tissue functions to constrict the veins in the corpus cavernosum. This in turn functions to maintain blood in the penis and facilitate erection. In operation therefore, the stimulator may be activated to cause stimulation of the contractile tissue 2 during or before sexual stimulation to promote an erection, maintaining the erection once sufficient blood has entered the penis 52. If the main blood vessels, such as the deep dorsal vein, do not retain blood in the penis, or there is a massive venous leakage condition, it is believed that the stimulation of the contractile tissue band 2 will assist in maintaining an erection. Activation may occlude the main veins and/or occlude a tissue bed in order to retain the blood within the penis 52.

The implantable stimulator 1, in this embodiment, includes electronic circuitry in the form of a signal generator which is mounted in a bio-compatible housing 4. A more detailed description of the stimulator of this embodiment will be given later on in this document.

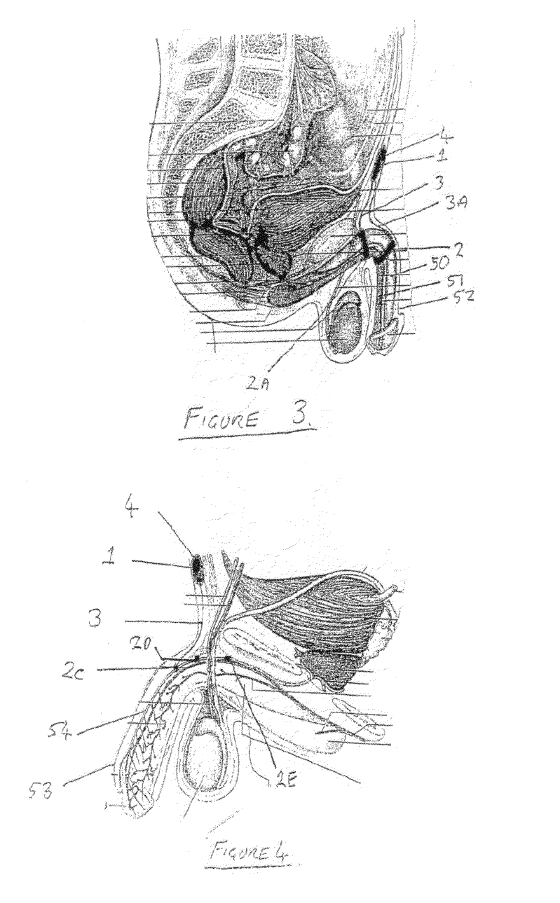

A further embodiment is illustrated in FIG. 2. In FIG. 2 the same reference numerals have been used for equivalent components to the FIG. 1 embodiment. In the embodiment of FIG. 2, the contractile tissue band 2 is implanted within the body, behind the root of the penis around the crura 53. The operation of this embodiment is similar to that of the embodiment of FIG. 1, merely the positioning of the contractile tissue being different.

Reference numeral 60 indicates in outline a further alternative position for the contractile tissue band. It is implanted further into the body around the ends of the crura 53.

Note there may be a number of positions where the band may be placed.

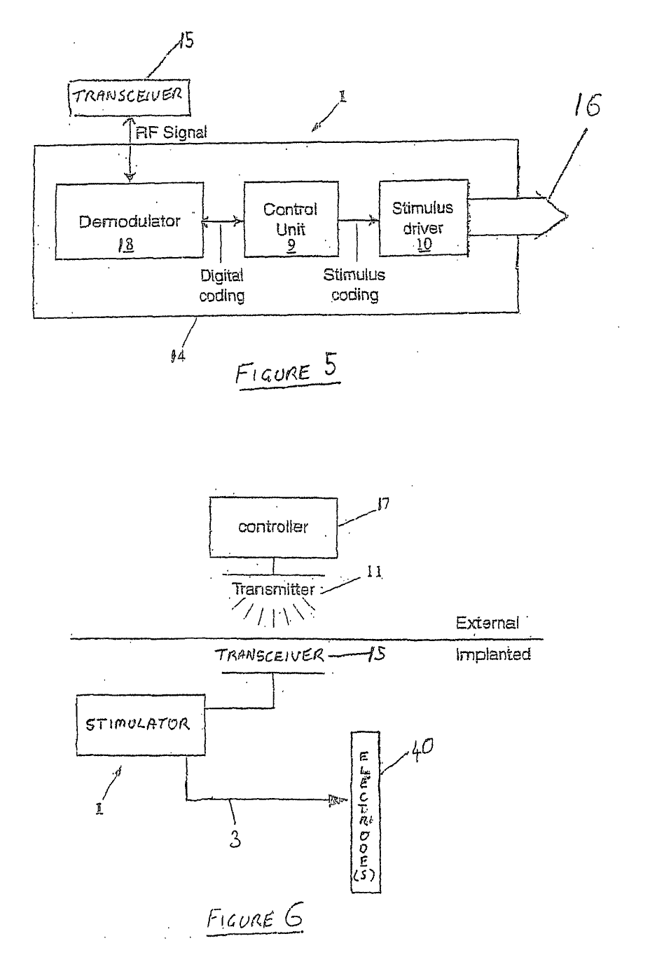

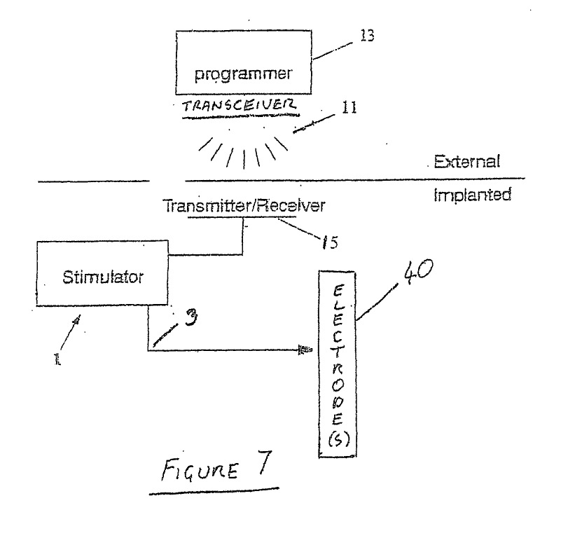

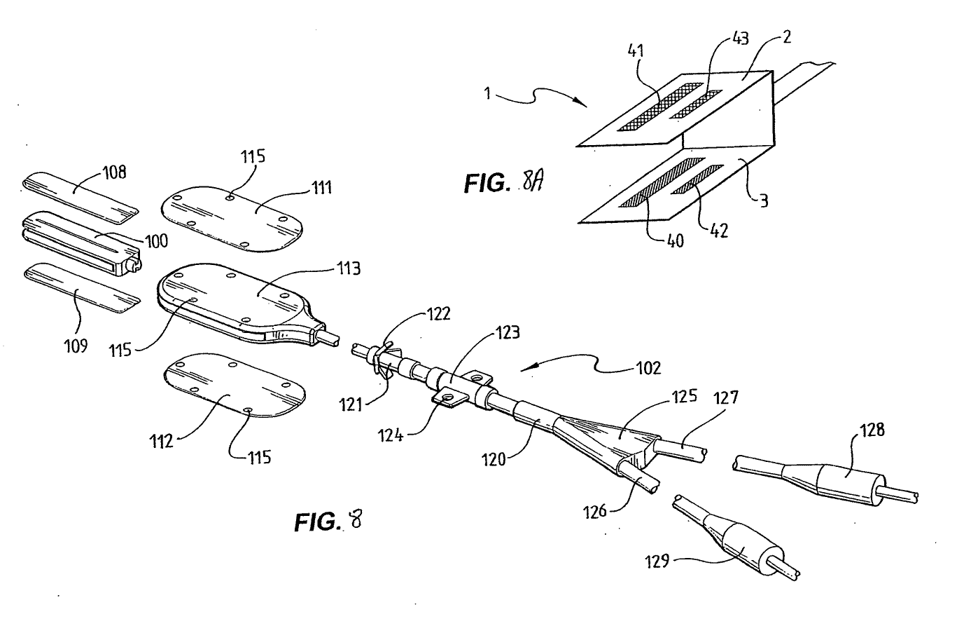

FIG. 3 illustrates yet a further embodiment. In this case there are two contractile tissue bands 2 and 2A. Band 2 is implanted in the same position as the band of the embodiment of FIG. 1 and band 2A is implanted in the same position as the embodiment of FIG. 2. A pair 3 and 3A of insulated conductors extend from the stimulator 4 to electrodes (not shown) implanted in the contractile tissue bands 2 and 2A. Both bands are therefore stimulated in use in order to facilitate erection.

The embodiment of FIG. 3 may be most useful where constriction along a substantial length of the penis is required in order to maintain an erection e.g. in severe cases of massive venous leakage.

Although two bands are illustrated in place in FIG. 3, there may be more than two bands in place at various positions, in alternative embodiments.

In a variation of this embodiment, the stimulator 4 may be arranged to selectively apply stimulation to one or other of the bands 2, 2A.

The dimensions of the implanted contractile tissue band in the embodiments of FIGS. 1 to 3 may be varied. Relatively narrow width bands are shown in the Figures, but the bands may be wider than this if deemed necessary to treat the condition. The depth of the bands will also be selected in order for optimal effectiveness.

In the above embodiments a band is wrapped around a substantial part of the penis e.g. around the crural bodies. Although assisting in occluding venous flow, this may also have an effect on arterial blood flow to the penis. In an embodiment, stimulation may be applied to the contractile tissue after arterial blood flow has been allowed for some time. That is, if there is poor arterial inflow, in this embodiment of the invention the timing of venous constriction can be manipulated to allow more filling time of the penis.

In other embodiments (see FIG. 4 below) the contractile tissue may take the form of a wrap or wraps to be wrapped around one or more blood vessels (veins) to specifically occlude particular blood vessel(s). In an embodiment, one or more blood vessels may be identified by a clinician as leaking more than the others and a contractile tissue wrap may be utilised to affect the particular blood vessels which are identified as most prone to leakage of blood.

In FIG. 4, a contractile tissue wrap 2C is implanted about the deep dorsal vein 54 of the penis 53. Stimulation applied to the wrap 2C by the stimulator 4 causes the wrap 2C to contract and occlude the deep dorsal vein. This therefore facilitates erection, particularly where there is a leakage problem with the dorsal vein. Two further alternative positions for the wrap are illustrated by reference numerals 2D and 2E.

Other blood vessels apart from the deep dorsal vein (although this is the major vein of the penis) may be treated in this way by implanting small contractile tissue wraps about the blood vessels to directly treat the leaking blood vessel itself. Other blood vessels that may be provided with a contractile tissue wrap include the Superficial Dorsal Vein, the Crural Tip Veins and the Cavernous Veins in the penile hilum. Using contractile tissue bands around only the affected blood vessels avoids any potential problem with using a large contractile band around the penis possibly occluding arterial blood flow to the penis.

Further, a combination of embodiments similar to FIGS. 1, 2 and 3 and the embodiment of FIG. 4 could be utilised in severe cases, so that a band of contractile tissues encircling the cavernosal bodies and blood vessels is provided, and one or more (smaller) wraps about particular blood vessels such as the deep dorsal vein are also provided.

In the embodiments discussed above, the contractile tissue is smooth muscle tissue. The smooth muscle tissue may be obtained from elsewhere in the body, formed into a wrap or band and surgically implanted. Alternatively, the smooth muscle tissue may be grown from the smooth muscle stem cells and/or proliferative smooth muscle cells. Alternatively the smooth muscle tissue may be transplanted smooth muscle tissue augmented by smooth muscle stem cells and/or proliferative smooth muscle cells. The smooth muscle tissue in this embodiment is innervated (by nerves growing into it over a period of time following the implantation) and stimulation by the electrodes stimulates the nerves in the smooth muscle to cause the smooth muscle to contract.

International Patent Application No. PCT/2006/001301 referred to above, discloses augmentation of contractile tissue using proliferative smooth muscle cells or smooth muscle stem cells. Growth, maturation and stability of the tissue may be influenced by growth factors (trophic and/or neurotrophic factors) that are a component of the treatment.

The smooth muscle may be taken from the smooth muscle of the bladder. Alternatively, the muscle is venous smooth muscle, or coccygeus smooth muscle, terminal ileum transplanted as a segment devoid of mucosa and having its circulation intact. A further alternative is the dartos smooth muscle from the scrotum. A further alternative may be a portion of conveniently located vein. In each case, the long axes of the muscle cells are disposed substantially circumferentially about the wrap/band. Depending upon the muscle selected, the circulation may or may not be transplanted intact. If the circulation is not transplanted intact, new vessels will need to be regrown, or otherwise provided.

In an embodiment, smooth muscle may be taken as a free graft. In this case, the tissue is separated from its normal circulation and becomes vascularised by ingrowth of blood vessels at the site of implant.

The contractile tissue need not be smooth muscle, but could be other types of contractile tissue. For example, skeletal muscle tissue could be utilised. The tissue could also be synthetic contractile tissue.

The stimulator 1 is shown in more detail in FIG. 5. In this embodiment, a signal generator that is arranged to provide the electrical signal for stimulation of the contractile tissue 2 is in the form of a control unit 9 and stimulus driver 10. The control unit 9 encodes the stimulus and provides a signal to the stimulus driver 10 which provides the stimulation signal at output 16. The output 16 outputs to conductor 3 and to one or more electrodes 40.

In this embodiment, the control unit 9 and stimulus driver 10 form, together with a demodulator 18, a processing unit for generating the stimulation signal(s) at output 16.

The demodulator 18 is arranged to demodulate a signal received by transceiver 15. An external control unit and external programmer unit (both to be described later) are able to communicate via the transceiver 15 with the processing unit 14 in order to control application of stimuli and/or vary the stimuli. In addition, as described in more detail later, the processing unit 14 may transmit, via control unit 9, demodulator 18 and transceiver 15, signals to a control unit or programmer unit. The transmitted signals may deliver telemetry information indicative of parameters of the stimulator, for the purposes of calibration and control.

The entire stimulator 1 (including components 14 and 15), is enclosed in a housing 4 which includes a casing made from a bio-compatible material, such as titanium, silicone polymer or other inert materials. The frequency of the RF signal for transmission and reception by the transceiver 15 may depend on the material of the casing of the stimulator. The stimulator is fully implanted.

FIG. 6 shows a system in accordance with an embodiment of the present invention. The system incorporates the implanted stimulator 1, with transceiver 15. The electrode(s) 40 is shown schematically together with cable 3.

The system also comprises an external controller 17 which includes a transmitter 11. The controller 17 is intended for operation by a patient with the stimulator implanted, for control of the stimulator 1.

The controller 17 includes means (such as a button, not shown) operable by the patient to selectively send signals to the implanted stimulator 1, for control of the stimulation signals being sent to the electrode(s) 40.

In this embodiment, no stimulation signals are sent until a signal is received from the controller 17. When the patient wishes to facilitate an erection (before or during sexual stimulation) they operate the controller 17 to send an “on” signal to the stimulator 1. In response to receiving the “on” signal, the controller unit 9 operates to turn the stimulating signal on, causing contractile tissue 2 to contract and facilitate and maintain an erection.

The controller 17 is also arranged to provide a further signal under patient control, once the patient no longer requires an erection, the further signal causing the stimulator 1 to stop providing the signal and to allow the contractile tissue to relax.

Where the contractile tissue is smooth muscle tissue, a stimulation signal 16 provided to contract the smooth muscle tissue 2 is selected so as to provide a substantially continuous tone in the smooth muscle. A charge-balanced biphasic pulse may be suitable for this. The signal has a substantially constant current less than or equal to 30 mA, and may be in the order of 15 mA. Stimulation pulse frequency provided to tissue 2 is in the range of 0.25 Hz to 2.5 Hz and is preferably 2 Hz. Stimulation pulse width is in the range of 0.05 m/s to 0.02 m/s and is preferably 0.15 m/s. The stimulator is current regulated, and accordingly the stimulation voltage will vary with the resistance of the muscle tissue between the electrodes. Typical values for the voltage are between 0.2 and 7 volts.

In a further embodiment, the stimulation signal may be a generally rectangular and symmetrically biphasic pulse. The signal has a substantially constant current less than or equal to 50 mA, 15 mA, 10 mA, or 5 mA, and in some preferred embodiments may be in the order of 4 mA, 8 mA, 12 mA, or 15 mA.

Stimulation pulse frequency provided to sphincter 1 may be in the range of 0.1 Hz to 5 Hz, 0.2 Hz to 4.0 Hz. 0.25 Hz to 3.0 Hz, 1 Hz to 3.0 Hz, 1.5 Hz to 3 Hz, 1.75 Hz to 2.5 Hz, or a 0.25 Hz to 2.25 Hz, and in one embodiment, is 1 Hz, 2 Hz, 2.5 Hz or 3 Hz. Stimulation phase width of each phase may be in the range of 0.05 ms to 2.0 ms, 0.1 ms to 1.5 ms, 0.2 ms to 1 ms, 0.25 ms to 0.75 ms, and in one embodiment is 0.2 ms, 0.4 ms, 0.5 ms or 1 ms. The stimulator is current regulated, and accordingly the stimulation voltage will vary with the resistance of the muscle tissue between the electrodes.

Typical values for the voltage may be between 0.1 and 15 Volts, 0.2 and 12 Volts, 0.5 and 12 Volts, 0.5 and 10 Volts, or 0.5 and 7.5 Volts. In one embodiment, the voltage is 2.5 Volts, 5 Volts, 7.5 Volts or 10 Volts. Either a current source (voltage limited) or a voltage source (current limited) stimulator may be used.

The controller unit may not only be a simple “on-off” device. It may enable the user to apply some control over the strength of the signal to be applied to the contractile tissue 2. This may enable the user to vary the extent of contraction of the tissue.

In an embodiment, the controller unit includes a controller enabling a user to vary the stimulation level for comfort and maintenance of an erection. For example, the controller may be used by the patient to increase stimulation if erection is not being maintained.

In one embodiment, the stimulator may default to the last stimulation level used to maintain the erection when it was last turned on.

In one embodiment, the stimulator may operate to slowly ramp up the stimulation so that there is less awareness of unwanted side effects by the patient. In another embodiment, this may not be required.

FIG. 7 shows a system in accordance with an embodiment of the present invention, including a programmer unit 13 which may be utilised by a physician to set and adjust parameters of the implanted stimulator 1. The programmer unit 13 is arranged to communicate with the stimulator via transceiver 11, and may include a computing device. The control unit 9 is also arranged to transmit stimulator telemetry information indicative of one or more of the parameters of the stimulator 1, for detection by the programmer 13 via transceiver 11. The programmer unit 13 can therefore determine parameters of the stimulator from telemetry information and can adjust the parameters by transmitting control signals to the stimulator 1. The signals from the programmer may be able to selectively vary the output current, shape, frequency and/or pulse width of the stimulation signal(s).

In operation, a physician adjusts parameters of the stimulation signal (s). The physician will note feedback from the patient as to the effect of the stimulus, and may subsequently re-adjust the parameters until the stimulation is optimum. For example, patient perceived feedback may be used to set the maximum stimulation threshold of a smooth muscle sphincter.

In the above-described embodiments, signals between the controller or programmer and the stimulator are RF signals. Other types of transmission media other than RF may be used. For example, microwave signals may be used for transmission, optical signals may be used, and in another embodiment magnetic transmission may be used.

Magnetic transmission may be used for the controller 17 to cause the stimulator to stop producing stimulation signals. In this embodiment, the controller 17 may be a simple magnet which, when passed over a magnetic receiver of the stimulator 1, results in the stimulator commencing to provide stimulation signals, and when passed over the stimulator 1 again, results in the stimulator ceasing to provide stimulation signals.

Other means than magnetic transmission may be utilised.

In the above embodiment, the implantable stimulator 1 is shown implanted within the patient in the abdomen. It may be implanted in any part of the patient that is convenient.

In the above embodiment, the stimulator is totally implantable. It is possible that the stimulator may be partly implantable and partly exterior. That is a signal generator could be placed exterior to cause an implanted conductor to conduct signals to the contractile tissue. The implanted conductor may include an electrode and an apparatus arranged to receive the stimulation signals from the external stimulator. The stimulator may be of any other configuration.

In the above embodiments, there may be one or more electrodes implanted in the contractile tissue at predetermined location(s). Types of electrode configurations that may be employed include bipolar electrodes, tripolar electrodes, cuff-type electrodes and other electrode types. In one embodiment, a “clothes peg” type electrode may be utilised, such as disclosed in applicants co-pending international patent application number PCT/AU2005/001698, referred to above. This type of electrode includes a pair of electrode elements extending parallel to each other and positionable either side of a contractile tissue band to provide stimulation to the portion of the band between the electrode elements.

Electrodes of a clothes peg type which may be used to stimulate the contractile tissue will now be described.

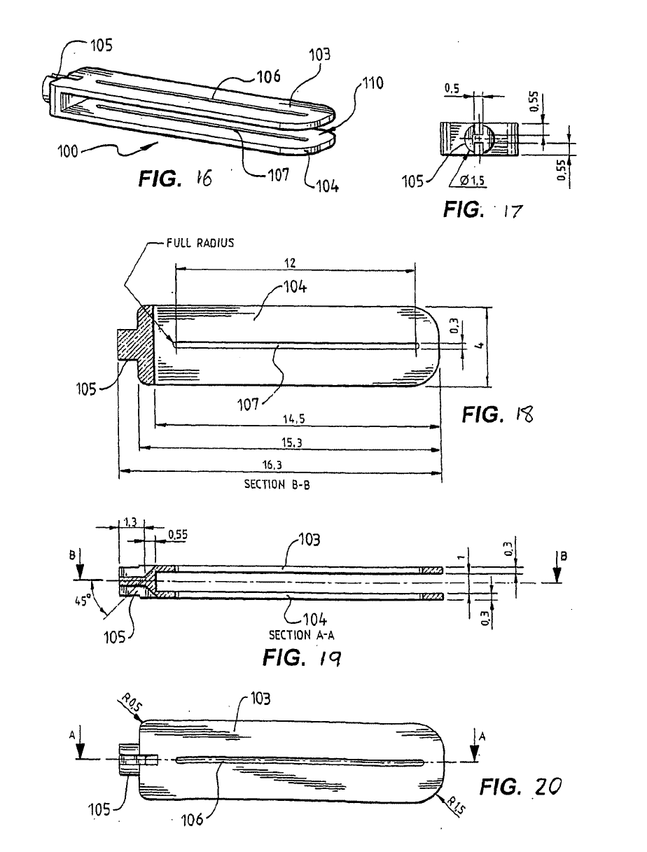

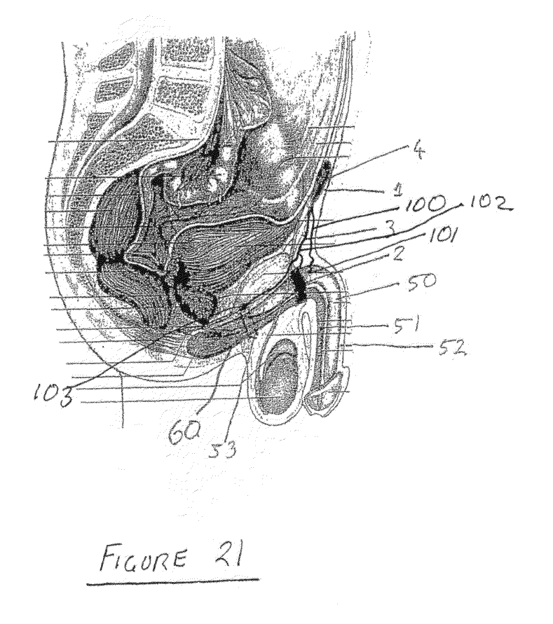

The electrode comprises a number of components. These include an electrode cover 100 (shown in most detail in FIGS. 15 through 20.

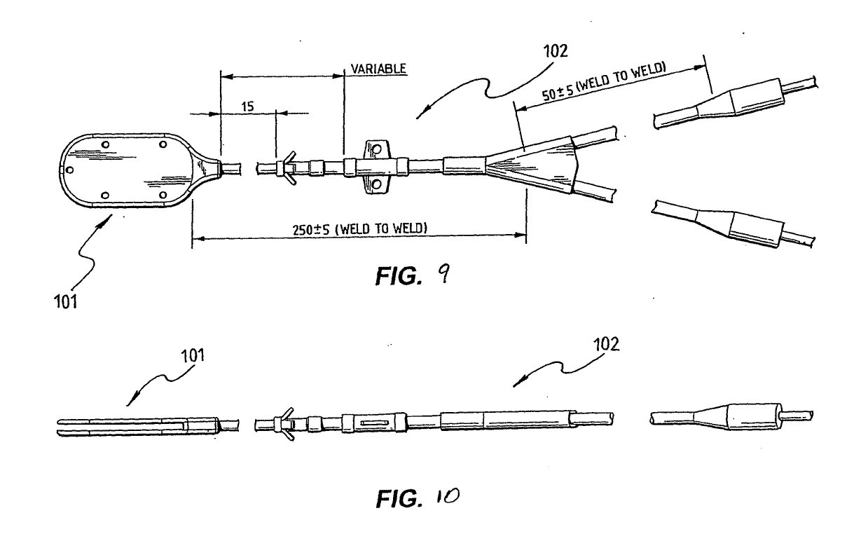

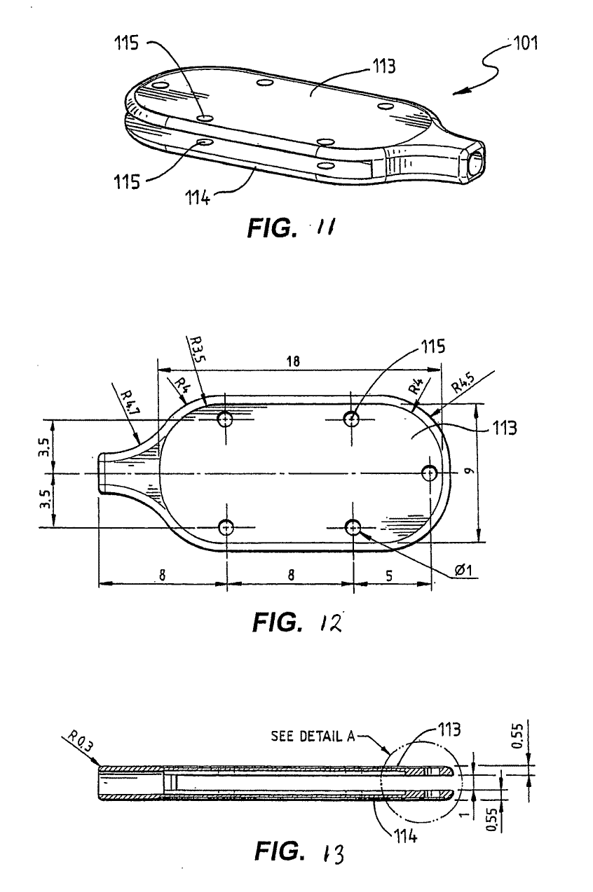

The components also include an electrode shroud (shown in best detail in FIGS. 11 through 14) and also an electrode lead 102 (shown in FIGS. 8, 9 & 10, together with the other components of the electrode arrangement).

In this embodiment first and second electrode elements are formed by the electrode cover 100, which includes insulating elements 103,104 extending from a base 105. The insulating extending elements 103,104 are formed with a slot 106,107, respectively, extending substantially along the length of the extending elements 103,104. When the electrode arrangement is assembled, platinum foil electrodes 108,109 (FIG. 8) are placed on the outer surfaces of the elements of the elements 103,104 so that they are insulated from the gap 110 formed between the elements 103,104 apart from the slots 106,107, which expose portions of the conductive plates 108,109 to the gap 110 (and, in use, to any tissue seated within the gap).

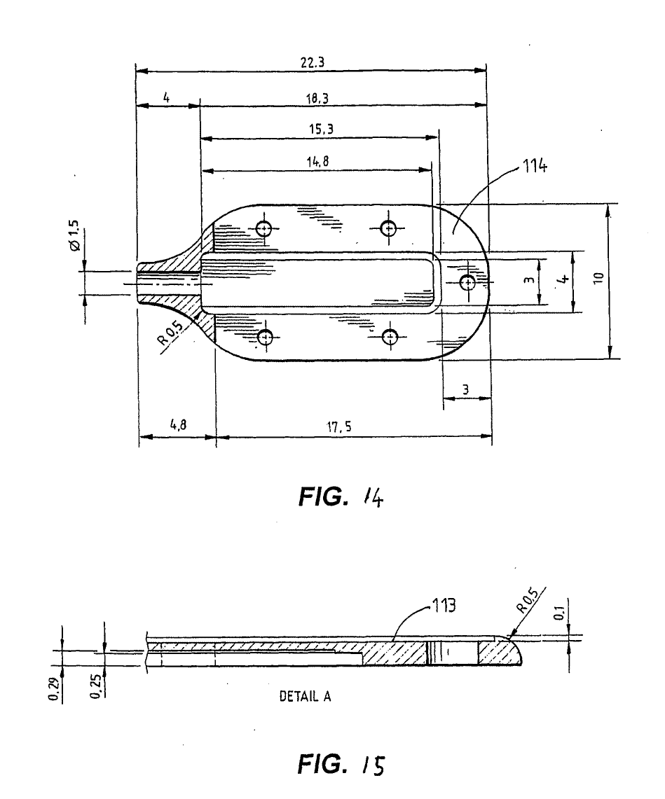

When assembled, the electrode cover 100 and platinum electrode foils 108,109 seat within the electrode shroud 101 as best shown in FIGS. 11, 12, 13 & 14. FIG. 14 in particular shown in cross-section where the electrode cover seats.

Electrode shroud 1 is formed from silicone. In order to provide reinforcement, PET mesh covers 111,112 are provided to fit to upper 113 and lower 114 extending portions of the shroud 101. Suture holes 115,116 are provided in the covers 111,112 and also in the elements 113,114 of the shroud 101. Note that the reinforcement can be provided by other means and is not limited to PET mesh. Further, the electrode shroud need not be in silicone but could be of other bio-compatible material and may not require re-inforcement. Further, note that other means for affixing to the tissue may be provided other than suture holes or instead of suture holes.

The electrode lead 102 is a multi-component arrangement which includes an outer insulating cover 120, a tine collar 121 including tines 122 for retaining the lead in position within a patient. It also includes a sutured collar 123 including suture holes 124 for suturing to patient tissue to also facilitate retaining the lead 102 in position. There is also bifurcation moulding 125 which enables the lead to split into two parts 126,127 which may contain separate conductors, and connectors 128,129 which may be arranged to contact to a simulation device.

In the above embodiments, the electrode arrangement includes a pair of electrode elements which extend away from a base which joins them together at their proximal ends. In a further embodiment, a single electrode element which is not joined at any base is provided. This single electrode element may be used to provide stimulation to contractile tissue on its own, or may be used together with one or more similar electrode elements to provide stimulation.

In the above described embodiments, each electrode element is provided with a single electrode. The single electrode is an elongate electrode extending substantially the majority of the length of the electrode element.

One advantage of having thin electrodes bounded by insulating material on either side is that the arrangement operates to confine the electric field produced by the electrode to the tissue immediately adjacent the electrode. This reduces or prevents stimulation of tissue that it is not desirable to stimulate eg. tissue external to a contractile tissue sphincter being controlled.

In operation, the electrodes 108, 109 and extending elements 103, 104 are positioned either side of the smooth muscle implant to enable signals to be transmitted to the implant for operation.

The electrode arrangement allows application of an electric field between the opposing electrode elements to stimulate the tissue between them. The electric field in one embodiment is confined so that stimulation is to a band of tissue between the electrodes.

In one embodiment, innervation runs within the implant 2 perpendicular to the band of tissue being stimulated.

The elements in the electrode extend over the tissue in a manner analogous to that of a clothes peg.

Other electrode patterns then a single line electrode on the surfaces of the elements may be utilised.

FIG. 8A discloses one alternate electrode pattern.

As discussed above, in an embodiment, the stimulater implant is preferably sealed and encased in a biologically inert material such as a biocompatible silicone material. Metallic electrodes and leads may be of plantinum-iridum alloy. The connecting wires are, in one embodiment, insulated with a silicon coating.

In a further embodiment of the present invention, a stimulator is also arranged to apply stimulation to a nerve or nerves as to affect arterial blood supply to the penis. This is advantageous where the patient also has a problem with arterial blood flow to the penis (as well as a problem with venous leakage).

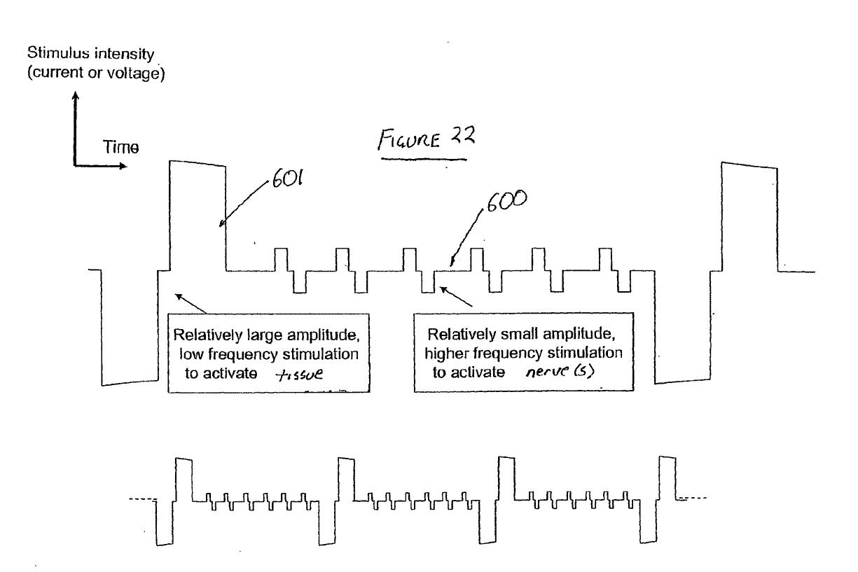

FIG. 21 illustrates a further embodiment of the invention. A further electrode lead 100 is illustrated in this embodiment, providing a signal to an electrode or electrodes 101 for stimulation of nerves associated with arterial function. Note the position of the electrode 101 is for the purposes of illustration only, and is not accurate. An electrode or electrodes may be placed in position which can affect arterial blood flow by a nervous stimulation, and the position will be selected by the clinician.

In this embodiment, the signal generator 4 also provides stimulation signals to the electrode(s) 101 for stimulation of arterial blood flow. Stimulation may be provided at the same time or slightly in advance of stimulation to the contractile tissue 2, in order to facilitate erection. The time, frequency and extent of stimulation to electrodes 101 will be determined by the clinician and will depend on the condition of the patient. In this embodiment, the control unit and programmer unit may include circuity for affecting control of the signal 100 for stimulating the electrode(s) 101.

In a further aspect of the invention, stimulation of the nerve or nerves to affect arterial blood flow may be carried out without stimulation of any contractile tissue to affect venous blood flow. That is, the arterial condition may be treated separately. In this embodiment, there may be no contractile tissue and no electrode or stimulation to any contractile tissue.

In accordance with a further embodiment, the stimulator 4 may also produce signals to stimulate nerves to affect other conditions that the patient may have. Such stimulation may be termed neuromodulation in which activity in one neural pathway modulates the pre-existing activity in another.

For example, the patient may also suffer from urinary incontinence, and a further stimulation signal may be provided by the stimulator 4 to stimulate one or more convenient sensory nerves to assist with the management of this urinary incontinence condition. This may particularly affect urge incontinence (although the embodiment is not limited to treating urge incontinence). The Dorsal Penile Nerve is a branch of the pudendal nerve, that originates from the sacral outflow from the spinal chord. Low levels of stimulation to the DPN may illicit neuromodulation effects to overcome an inappropriate urge to urinate. In an embodiment, stimulation to the contractile tissue may also deliver stimulation to the Dorsal Penile Nerve.

Neuromodulation is not limited to the Dorsal Penile Nerve, however, and other nerves in the anatomy may be stimulated. FIG. 21 illustrates an example an electrode lead 102 from a stimulator 4 travelling to an electrode 103 for stimulation of nerves within the patient's pelvic anatomy.

Neuromodulation may also be under patient control via the controller and clinician control via the programmer unit.

In operation, neuromodulation may be carried out by the Dorsal Penile Nerve which is convenient to the site of placement of the smooth muscle wrap, or, alternatively, any other nerve that is appropriate to treat urge incontinence. A neuromodulating effect may also occur at the same time as the signals being provided to the contractile tissue to maintain an erection. Alternatively neuromodulation may be turned off when the signal is provided to the contractile tissue.

A single electrical signal of a predetermined pattern may be used to stimulate the contractile tissue and the one or more nerves. In other embodiments, however, separate signals may be provided for neuromodulation and stimulation of the contractile tissue.

The step of applying neuromodulation to stimulate the one or more nerves may comprise initially applying electrical signals at relatively low level and increasing to the required level. This “ramping up” may reduce annoying perception of the stimulation by the patient. Otherwise they may perceive this stimulation as unpleasant tingling, for example.

The stimulation of the contractile tissue may be by relatively low frequency signals for stimulation of the contractile tissue, and stimulation of one or more nerves for neuromodulation by a relatively high frequency signal. The low frequency signal may be from 1 Hz to 5 Hz and the relatively high frequency signal 5 Hz to 200 Hz.

A relatively low intensity signal may be utilised for neuromodulation and a relatively high intensity signal for stimulation of the contractile tissue.

A plurality of electrodes placed in various positions may be used to provide the neurostimulation. Each electrode may deliver a distinct electrical signal to a plurality of different sites in order to effect the required nerve stimulation. As discussed above, the same signal that is used to stimulate the contractile tissue may be used to provide the neurostimulation. This is unlikely, however, as different properties may generally be required of the signals. In an embodiment, therefore, either separate signals are provided (separate channels) or the signals may be interleaved. In either case the stimulation parameters are distinct for the different purposes of (i) stimulating the contractile tissue and (ii) stimulating one or more nerves e.g. Dorsal Penile Nerve.

FIG. 13 shows a diagram of an example of an interleaved-type signal. The signal 600 for stimulation of the one or more nerves has a relatively small amplitude and a higher frequency. The signal 601 for stimulating the contractile tissue, has a relatively large amplitude and low frequency.

A relatively high frequency signal may be used to provide the stimulation for the nerves and a relatively low frequency signal to stimulate the tissue. The relatively high frequency signal may be greater than 5 Hz, preferably greater than 8 Hz and even more preferably 10 Hz or greater. In one embodiment, the high frequency signal is up to 100 Hz.

The low frequency signal, for stimulation of the tissue, will usually be less than 5 Hz and may be 2 Hz or less.

A relatively low intensity signal may be used to stimulate the nerves and a relatively high intensity signal for the tissue. The low intensity signal in this embodiment is a pulse signal. The pulse signal may have 300 us or less pulse width and a 3 mA or less current, and in this embodiment 200 us or less and 1 mA current.

The high intensity tissue stimulating signal may have pulse width greater than 300 us and a current of greater than 3 mA, and in this embodiment is 400 us or more and the current is 4 mA.

The duty cycle for the nerve stimulating signal may vary. As discussed above in this embodiment it is 5 minutes on and 5 minutes off but in other embodiments may be 5 minutes or more on and 15 minutes or more off, 5 minutes or less off and 5 minutes or less on. Duty cycle and other signal characteristics may be adjusted to provide the most effective stimulation. In some embodiments, the duty cycle may be in terms of seconds (e.g. less than 10 seconds on and less than 30 seconds off).

With the nerve stimulation signal, it is possible that the patient may experience some discomfort (e.g. ‘tingling’) on application of the signal. Where the signal is applied in accordance with a duty cycle, this tingling may be experienced in rhythm with the duty cycle. In one embodiment, the afferent nerve stimulating signal may be “ramped up” from a relatively low intensity to the required intensity each time it is applied. This may reduce unwanted awareness of the stimulation or patient discomfort.

The electrodes employed may incorporate an electrically conductive surface that is in contact with the contractile tissue, which also activates afferent nerve fibres. In another embodiment, the electrode for the tissue may include one or more additional electrically conductive surfaces that are on the outer surface of the electrode, to stimulate nerve endings in the surrounding anatomy. In yet a further embodiment, the electrode may be entirely separate to the electrode used to stimulate the tissue, but placed conveniently in the adjacent anatomy to facilitate delivery of electrical stimulation to neurostimulate the bladder reflexes.

Neuromodulation may be used to treat other conditions in urge incontinence. It may be used to treat fecal incontinence, for example.

The interleaved signals shown in FIG. 22 may not operate at the same time. For example, signal 601 may only be required to operate when erection maintenance is required.

Embodiments of the present invention may be utilised not only to treat conditions of erectile dysfunction brought about by venous leakage or massive venous leakage. They may assist in treatment for psychological conditions, for example, where it is difficult to maintain an erection because of a psychological difficulty. It is envisaged that embodiments may even be utilised where there is no particular erectile dysfunction condition. For example, to enhance sexual performance e.g. by maintaining erections for longer periods of time.

Positioning of the contractile tissue bands of the embodiments of FIGS. 1 to 3 may also be governed by aesthetic requirements. Positioning of a band of contractile tissue about the base of the penis, for example, may effectively increase girth of the penis.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An apparatus for facilitating an erection in a patient, the apparatus comprising a stimulator arranged to provide a signal for stimulation of contractile tissue, in order to facilitate an erection, the contractile tissue on contraction is arranged to constrict one or more blood vessels and/or the cavernosal bodies.

2. (canceled)

3. An apparatus in accordance claim 1, wherein the contractile tissue is implanted in the form of a band about the root of the penis.

4. An apparatus in accordance with claim 1, wherein the contractile tissue is implanted as a band within the body about the left and right crura.

5. An apparatus in accordance with claim 1, the contractile tissue being placed about one or more blood vessels.

6. An apparatus in accordance with claim 5, wherein the contractile tissue is implanted as a wrap about the deep dorsal vein.

7. An apparatus in accordance with claim 1, wherein the contractile tissue is smooth muscle tissue.

8. An apparatus in accordance with claim 1, wherein the contractile tissue is skeletal muscle.

9. An apparatus in accordance with claim 1, wherein the signal is in the form of a pulse signal, arranged to maintain tone in the contractile tissue.

10. An apparatus in accordance with claim 1, the stimulator being arranged to provide a different stimulation signal or no stimulation signal in order to allow the contractile tissue to relax.

11. An apparatus in accordance with claim 1, the stimulator being further arranged to stimulate a nerve or nerves affecting arterial blood supply to the penis.

12. An apparatus in accordance with claim 1, the stimulator being arranged to provide a signal to stimulate one or more nerves in the patient's anatomy to treat another condition.

13. An apparatus in accordance with claim 12, wherein the condition is urinary incontinence.

14. An apparatus in accordance with claim 13, wherein the condition is urge incontinence.

15. An apparatus in accordance with claim 12, wherein the condition is fecal incontinence.

16. An apparatus in accordance with claim 12, wherein the one or more nerves comprises the Dorsal Penile Nerve.

17. A device for facilitating an erection, the device including implanted contractile tissue positioned proximate or within the penile anatomy and arranged to be stimulated to contract to facilitate an erection, the contraction being arranged to constrict one or more blood vessels and/or the cavernosal bodies.

18-33. (canceled)

34. A method of facilitating an erection, comprising the steps of stimulating contractile tissue positioned proximate or in the penile anatomy of a patient in order to cause the contractile tissue to contract and constrict one or more blood vessels and/or the cavernosal bodies, by way of providing a stimulation signal to an electrode arranged to transmit the signal to the contractile tissue.

35. A method in accordance with claim 34, comprising the further step of providing a further signal, or absence of a signal, in order to enable the contractile tissue to relax.

36. A method in accordance with claim 34, comprising the further step of providing a signal for stimulation of a nerve or nerves affecting arterial blood supply to the penis.

37. A method in accordance with claim 34, further comprising the step of providing a signal to stimulate one or more nerves in a patient's anatomy to treat another condition.

38. A method in accordance with claim 37, wherein the condition is urinary incontinence.

39. A method in accordance with claim 38, wherein the condition is urge incontinence.

40. A method in accordance with claim 37, wherein the condition is fecal incontinence.

41. A method in accordance with claim 37, wherein the one or more nerves comprises the Dorsal Penile Nerve.

42-60. (canceled)
